(19) United States
(12) Patent Application Publication
(10) Pub. No.: US 2019/0293664 A1
(43) Pub. Date: Sep. 26, 2019

(54) METHOD FOR INDIRECTLY MEASURING LEAD ION CONCENTRATION IN BLOOD

(71) Applicant: KAOHSIUNG MEDICAL UNIVERSITY, Kaohsiung (TW)

(72) Inventors: Yu-Chang TYAN, Kaohsiung (TW); Hung-Yi CHUANG, Kaohsiung (TW); Ming-Hui YANG, Kaohsiung (TW)

(21) Appl. No.: 15/928,573

(22) Filed: Mar. 22, 2018

(51) Int. Cl. G01N 33/72

(52) U.S. Cl. CPC G01N 33/72; G01N 30/7233

(57) ABSTRACT

A method for measuring blood lead ion concentration comprising the steps of: providing a blood sample; analyzing the blood sample by using a mass spectrometry to obtain a spectrum; calculating an intensity area of a characteristic peak at mass-to-charge ratio (m/z)=1088.16±0.05 in the spectrum; and calculating a lead ion concentration (μg/dL) in the blood sample using the formula of y=0.875x+11.5, wherein y indicates the intensity area, and x indicates the lead ion concentration in the blood sample.

### FIELD

The present invention relates to a method for measuring blood lead ion concentration. More specifically the present invention relates to a method for indirectly measuring blood lead ion concentration.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Conventionally blood lead ion concentration is directly converted from the signal intensity the lead ions represent. Inductively coupled plasma-mass spectrometry (ICP-MS) analysis is a common measurement. However, before analyzing, the blood sample has to be treated with a strong acid (e.g. nitric acid) digestion and high temperature drying, leading to operational risks. In addition, strong acids and high temperatures are harmful to the environment. Moreover, the conventional method may not work effectively if the volume of blood is insufficient (required blood volume: at least 10 mL).

### SUMMARY

It is an objective of the present invention to overcome the problems of the conventional method for measuring blood lead ion concentration.

The present invention provides a method for measuring blood lead ion concentration comprising the following steps: providing a blood sample; analyzing the blood sample by using mass spectrometry to obtain a spectrum; calculating an intensity area of a characteristic peak at mass-to-charge ratio (m/z)=1088.16±0.05 in the spectrum; and calculating a lead ion concentration (μg/dL) in the blood sample using the formula of y=0.875x+11.5, wherein y indicates the intensity area, and x indicates the lead ion concentration in the blood sample.

In an embodiment of the present invention, the blood sample is a whole blood sample or a red blood cell sample.

In an embodiment of the present invention, the blood sample is treated with a digestive enzyme.

In an embodiment of the present invention, the digestive enzyme is trypsin.

In an embodiment of the present invention, the mass spectrometry is a liquid chromatography mass spectrometry (LC-MS) or a matrix assisted laser desorption ionization time-of-flight mass spectrometry (MALDI-TOF-MS).

In an embodiment of the present invention, the method of intensity area calculation is integration.

In an embodiment of the present invention, the intensity value of the characteristic peak is a signal intensity value presented by the amino acid fragment of SEQ ID NO: 1.

In an embodiment of the present invention, the amino acid fragment is derived from hemoglobin.

In an embodiment of the present invention, the lead ion is divalent lead ion, trivalent lead ion, or the combination thereof.

In an embodiment of the present invention, the lead ion concentration in the blood sample corresponds to the lead ion concentration measured by ICP-MS in the same blood sample.

The intensity area of a characteristic peak at mass-to-charge ratio (m/z)=1088.16±0.05 is not the signal intensity value presented directly by lead ion concentration. Therefore, the method of the present invention is different from the conventional method. Besides, the blood samples do not have to be treated with a strong acid or a high temperature, making the method relatively safe. Furthermore, the method only requires a small amount of blood sample, hence can reduce the limitations caused by blood volume.

These and other aspects of the present invention will become apparent from the following description of the preferred embodiment taken in conjunction with the following drawings, although variations and modifications therein may be effected without departing from the spirit and scope of the novel concepts of the disclosure.

### BRIEF DESCRIPTION OF THE DRAWINGS

The accompanying drawings illustrate one or more embodiments of the disclosure and together with the written description, serve to explain the principles of the disclosure. Wherever possible, the same reference numbers are used throughout the drawings to refer to the same or like elements of an embodiment, and wherein:

FIG. 1 is a MALDI-TOF-MS spectrum of the second group of a digested standard sample;

FIG. 2 is a comparison chart of the intensity area of a specific characteristic peak of the digested standard sample in the second group, and the intensity area of the same specific characteristic peak of the digested standard sample in the first group;

FIG. 3 is a liquid chromatograph tandem Fourier-transfer high resolution ion trap mass spectrometer (LC-MS/FT-HR ITMS) spectrum of the digested standard sample in the second group;

FIG. 4 is a comparison chart of the intensity area of a specific characteristic peak of standard samples with different digestive treatments;

FIG. 5 is a comparison chart of the intensity areas of a specific characteristic peak of digested subject's blood samples in different groups; and

FIG. 6 is a relation diagram of the relationship between the blood lead ion concentration and the intensity area of a specific characteristic peak of the digested subject's blood samples in different groups.

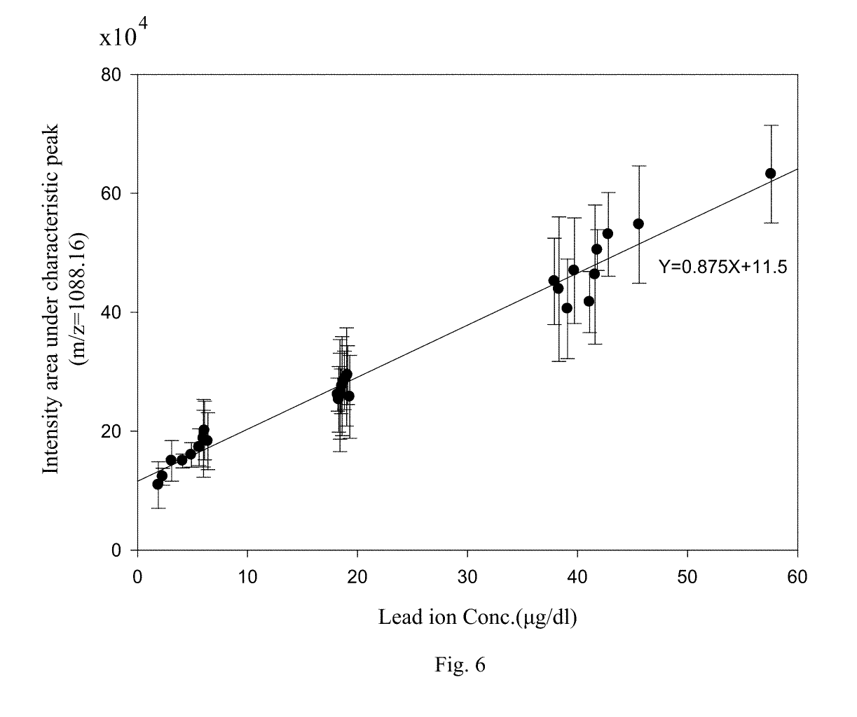

### DETAILED DESCRIPTION

In order to achieve the above objects and more, the following technical means and structures of the present invention are illustrated by drawings and described below. It should be noted that the described embodiments are illustrative and do not limit the present invention.

### Embodiment 1: Standard Sample Preparation

Hemoglobin solution preparation: the commercial hemoglobin standard was mixed with distillation-distillation H₂O (ddH₂O). Lead (II) acetate solution preparation: lead (II) acetate, normal saline, and 10% formic acid solution were mixed together.

First group of standard sample preparation: the hemoglobin solution was mixed with normal saline to get a mixture, and the mixture was then incubated in a 37° C. water bath for at least 12 hours. Second group of standard sample preparation: the hemoglobin solution was mixed with lead (II) acetate solution to get a mixture, and the mixture was then incubated in a 37° C. water bath for at least 12 hours.

### Embodiment 2: Standard Sample Digestion

One M dithiothreitol (DTT) solution was prepared with 25 mM ammonium bicarbonate; and 1M iodoacetamide (IAA) solution was prepared with 25 mM ammonium bicarbonate. The standard samples (100 μL) were respectively mixed with 1M DTT solution (10 μL) to get mixtures, and the mixtures were incubated in a 37° C. water bath for 3 hours. The mixtures were then respectively added with a 1 M IAA solution (10 μL) and reacted at room temperature in the dark for 30 minutes. Then the mixtures were added with 0.1 μg/μL trypsin and placed in 37° C. to hydrolyze (digest) the hemoglobin. Lastly, 2 μL formic acid was added to terminate the hydrolysis.

### Embodiment 3: Mass Spectrometry Analysis of Digested Standard Samples

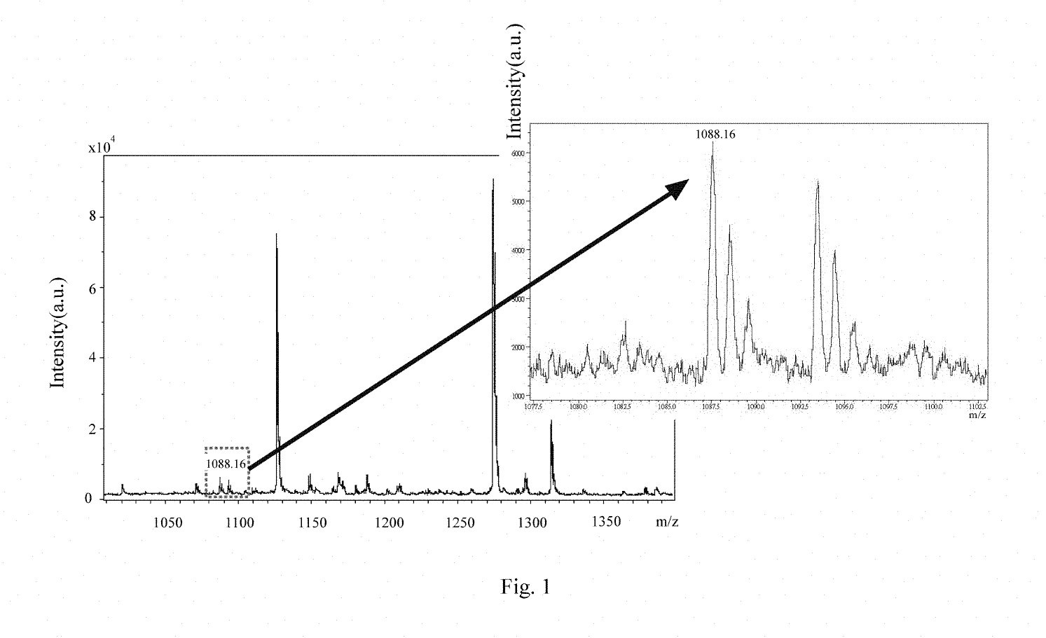

MALDI-TOF-MS was used to analyze the digested standard samples in each group. After identification of 634 spectrum signals, 29 signals were identified as hemoglobin fragments, and one signal, which was located at the highest peak of the characteristic peak at mass-to-charge ratio (m/z)=1088.16, of the 29 signals from different samples had significantly different signal intensities. FIG. 1 shows this signal intensity of the spectrum captured from the digested standard sample in the second group.

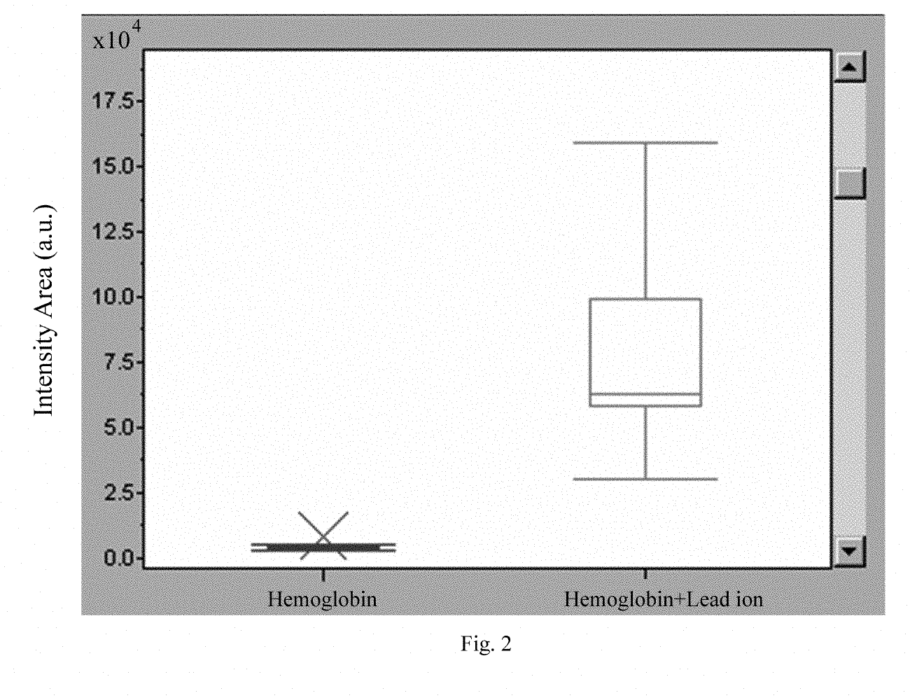

Next, integral software was used to calculate the intensity areas of this spectrum signal from the different samples. As shown in FIG. 2, the intensity area from the digested standard sample in the second group is higher than from the digested standard sample in the first group. The result indicates that the intensity area of the highest peak as the characteristic peak at mass-to-charge ratio (m/z)=1088.16 is positively correlated to the lead ion concentration in the samples.

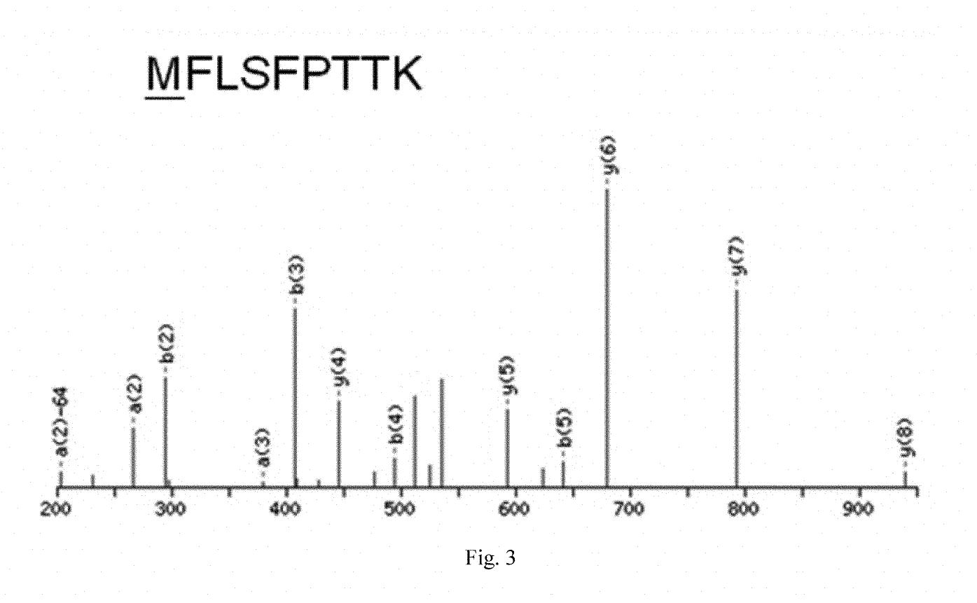

After that, the sequence in hemoglobin corresponding to this signal was analyzed by using a liquid chromatograph tandem Fourier-transfer high resolution ion trap mass spectrometer (LC-MS/FT-HR ITMS) spectrometry. As shown in FIG. 3, the sequence is MFLSFPTTK, wherein the underlined amino acid (Methionine) is oxidative. More specifically, the lead ion concentration in blood is proportional to the oxidation degree of methionine.

### Embodiment 4: Spectral Analysis of the Digested Blood Samples

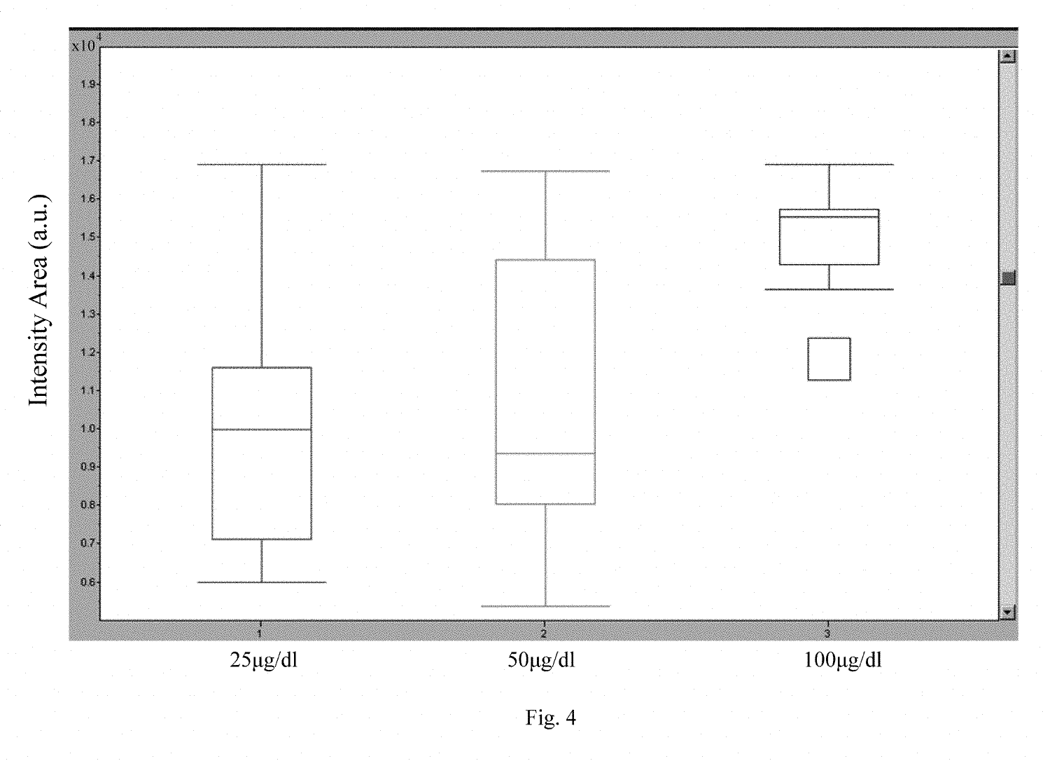

In order to validate whether the above described phenomenon exists in human blood or not, a human blood sample was collected and added with different concentrations of lead (II) acetate solution, and then treated with the above described digestion. The digested blood sample was then analyzed by MALDI-TOF-MS and integration software was used to calculate the intensity areas of the spectrums (the characteristic peak at mass-to-charge ratio (m/z)=1088.16) from different samples. As shown in FIG. 4, the intensity area of the characteristic peak at mass-to-charge ratio (m/z)=1088.16 is positively correlated to the lead ion concentration in the human blood sample.

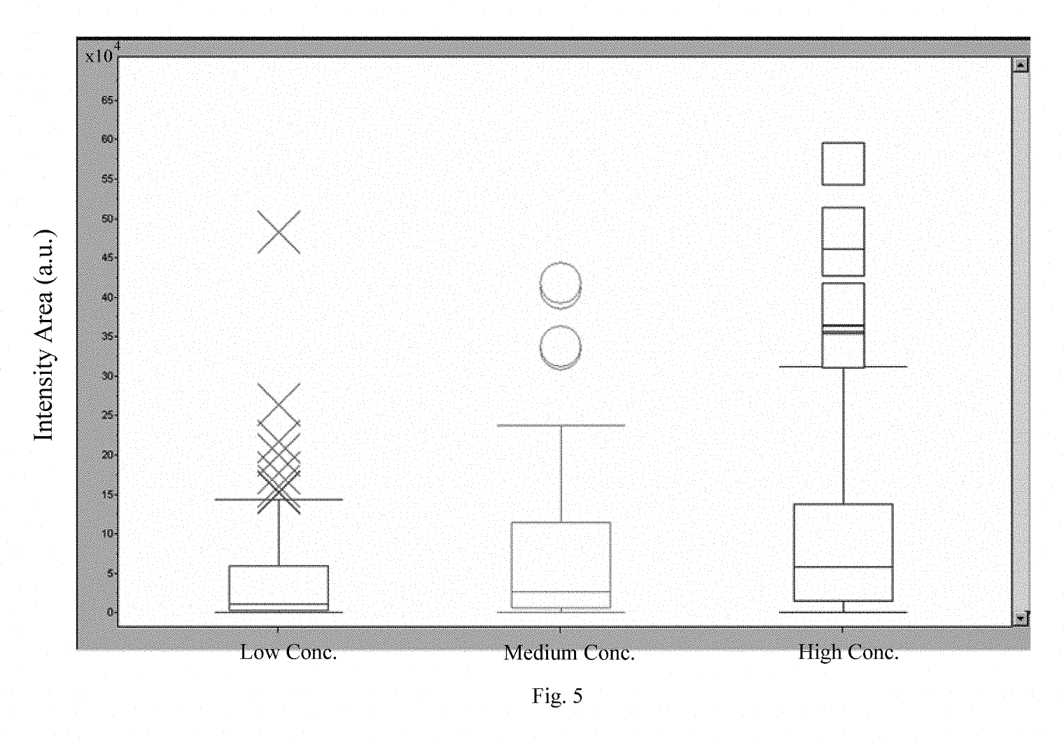

Blood samples were collected from the subjects in another group to analyze the lead ion concentrations in each blood sample by ICP-MS. The subjects were divided into three groups as a high concentration group, a medium concentration group, and a low concentration group according to the measured lead ion concentrations. Then, all blood samples were treated with the above described digestion. The digested blood sample was then analyzed by MALDI-TOF-MS, and the intensity areas of the spectrum signal (the highest peak as the characteristic peak at mass-to-charge ratio (m/z)=1088.16) from digested blood samples in each group were calculated by integration software. As shown in FIG. 5, the intensity area of this signal in the spectrum is positively correlated to the lead ion concentration in blood sample in different groups.

Last, a linear equation of y 0.875x+11.5 was established according to the intensity area of the characteristic peak at mass-to-charge ratio (m/z)=1088.16 and the lead ion concentration measured by ICP-MS. The y indicated the intensity area, and the x indicated the lead ion concentration in the blood sample. In this way, the intensity area can be converted to lead ion concentration in a blood sample.

Although the present invention is disclosed above by feasible preferred embodiments, the preferred embodiments are not restrictive of the claims of the present invention. Equivalent implementation and changes made by persons skilled in the art to the preferred embodiments without departing from the spirit of the present invention must be deemed falling within the scope of the present invention.

What is claimed is:

1. A method for measuring blood lead ion concentration comprising:
providing a blood sample;
analyzing the blood sample by using a mass spectrometry to obtain a spectrum;
calculating an intensity area of a characteristic peak at mass-to-charge ratio (m/z)=1088.16±0.05 in the spectrum; and
calculating a lead ion concentration (m/dL) in the blood sample using formula: y=0.875x+11.5,
wherein y indicates the intensity area, and x indicates the lead ion concentration in the blood sample.

2. The method according to claim 1, wherein the blood sample is a whole blood sample or a red blood cell sample.

3. The method according to claim 1, wherein the blood sample is treated with digestive enzyme.

4. The method according to claim 3, wherein the digestive enzyme is trypsin.

5. The method according to claim 1, wherein the mass spectrometry is a liquid chromatography mass spectrometry (LC-MS) or a matrix assisted laser desorption ionization time-of-flight mass spectrometry (MALDI-TOF-MS).

6. The method according to claim 1, wherein the intensity area is calculated by integration.

7. The method according to claim 1, wherein the intensity value of the characteristic peak is a signal intensity value presented by an amino acid fragment of SEQ ID NO:1.

8. The method according to claim 7, wherein the amino acid fragment is derived from hemoglobin.

9. The method according to claim 1, wherein the lead ion is a divalent lead ion, a trivalent lead ion, or a combination thereof.

10. The method according to claim 1, wherein the lead ion concentration in the blood sample corresponds to the lead ion concentration measured by inductively coupled plasma-mass spectrometry (ICP-MS) in the same blood sample.


SEQUENCE LISTING (1 sequence)

SEQ ID NO: 1 - protein, 9 aa, Homo sapiens
MFLSFPTTK